(19) United States
(12) Patent Application Publication
(10) Pub. No.: US 2009/0318346 A1
(43) Pub. Date: Dec. 24, 2009

(54) BIVALENT ErbB LIGAND BINDING MOLECULES AND METHODS FOR THEIR PREPARATION AND USE

(75) Inventors: Sarah S. Bacus, Hinsdale, IL (US); Jason E. Hill, Hinsdale, IL (US); Josef Yarden, Rehovot, IL (US); Bose S. Kochupurakkal, Bangalore (IN)

Correspondence Address: K&L Gates LLP, P.O. Box 1135, CHICAGO, IL, 60690, US

(73) Assignees: Targeted Molecular Diagnostics, Westmont, IL (US); Yeda Research and Development Co., Ltd., Rehovot (IL)

(21) Appl. No.: 12/278,751

(22) PCT Filed: Feb. 8, 2007
(86) PCT No.: PCT/US07/61863

§ 371 Date: Jun. 23, 2009

Related U.S. Application Data
(60) Provisional application No. 60/771,237, filed on Feb. 8, 2006.
(60) Provisional application No. 60/828,343, filed on Oct. 5, 2006.

(51) Int. Cl. A61K 38/16; C07H 21/04; A61P 35/00; C07K 14/00; C12N 5/10; C12N 1/19; G01N 33/574

(52) U.S. Cl. 514/12; 536/23.1; 530/350; 435/325; 435/254.2; 435/358; 435/7.23

(57) ABSTRACT

A new bivalent ErbB-based ligand binding molecule is disclosed along with its method of preparation and use. The binding molecule can be a protein expressed from a recombinant DNA molecule. The protein can contain two extracellular domains of an ErbB receptor that both bind to ErbB receptor ligands. These binding molecules act as traps to bind and sequester ligands, thus making them unavailable for binding to cellular ErbB receptors.

### BACKGROUND

Receptor tyrosine kinases are involved in stimulating the growth of many cancers. In general, receptor tyrosine kinases are glycoproteins which consist of (1) an extracellular domain that is able to bind with a specific ligand, (2) a transmembrane region, (3) a juxtamembrane domain where the receptor may be regulated by, for instance, protein phosphorylation, (4) a tyrosine kinase domain that is the enzymatic component of the receptor, and (5) a carboxyterminal tail. For many solid tumors, the ErbB family of type I receptor tyrosine kinases constitute one important class of receptors because of their importance in mediating cell growth, differentiation and survival. Members of this receptor family include ErbB1 (also known as HER1), ErbB2 (HER2/neu), ErbB3 (HER3), and ErbB4 (HER4). These receptor tyrosine kinases are widely expressed in a variety of tissues including epithelial, mesenchymal, and neuronal tissues. Overexpression of ErbB2 or ErbB1 has been correlated with a poorer clinical outcome in some breast cancers and a variety of other malignancies.

In their inactive state, ErbB receptors are generally thought to exist as monomers. Upon binding with their respective ligands, conformational changes can occur within the receptor which can result in the formation of receptor homo- and heterodimers, i.e., the activated receptor form. Ligand binding and subsequent homo- or heterodimerization can stimulate the catalytic activity of the receptor through autophosphorylation and transphosphorylation, that is, the individual monomers will phosphorylate each other on tyrosine residues. This can result in further stimulation of receptor catalytic activity. In addition, some of the phosphorylated tyrosine residues provide a docking site for downstream signaling molecules.

Activation of ErbB receptors can result in any of a variety of distinct effects such as proliferation and cell survival. These different outcomes occur through different signaling pathways that depend on the particular ligands that bind to particular receptors. Ligand binding dictates the population of the homo- or heterodimers that ultimately are formed. Numerous studies have shown that the type of bound ligand, and subsequent type of homo- or heterodimer formed, results in the differential phosphorylation of tyrosine residues on the activated ErbB receptors. As an example, the neuregulins (“NRGs”, also known as heregulins) are a family of ligands that can bind to ErbB receptors and elicit a variety of responses including proliferation, differentiation, survival, and migration. NRG1β and NRG2β can bind to ErbB3 and induce ErbB2/ErbB3 heterodimers, however, only NRG1β stimulates differentiation of breast cancer cells in culture. The reason for this is the recruitment of different downstream signaling molecules to the activated ErbB2/ErbB3 heterodimers when NRG1β is bound as compared to when NRG2β is bound. For example, although NRG1β and NRG2β result in similar overall levels of ErbB2 tyrosine phosphorylation, only NRG1β resulted in binding of PI3K (p85), SHP2, Grb2, and Shc to the receptor.

Current receptor tyrosine kinase based therapeutics generally fall into two categories. Small molecule inhibitors, such as Lapatinib, bind to the intracellular tyrosine kinase region and prevent ATP binding and receptor phosphorylation. A second type of therapeutic is based on monoclonal antibodies, such as Herceptin (Trastuzumab), that recognize and bind to the extracellular ligand binding domain of a particular receptor triggering receptor degradation. Both types of therapies have shown efficacy. However, it is clear that a variety of factors influence the relative efficacy of each therapy. For example, high levels of IGF-1R are known to interfere with Herceptin™ treatment, but not Lapatinib treatment. While different in their mechanisms of action, both Herceptin and Lapatinib target and bind to the receptors.

It is also becoming clear that overexpression of activating ligands can cause uncontrolled cellular proliferation similar to that of a deregulated receptor. In such cases, interference with the binding of the activating ligand to its receptor may provide a new therapeutic strategy that could be more effective or could accentuate current receptor based or other therapies alone.

Therapeutics that interfere with ligand binding to ErbB3 may be particularly effective. ErbB3 differs from the other receptors in the EGFR family because its tyrosine kinase domain is functionally inactive; however, ErbB2/ErbB3 hetrodimers transmit the most potent mitogenic signals of any homo- or heterodimer combination of the ErbB family. Therefore, ErbB3 is an important target, yet one that cannot be inhibited through small molecules that target the kinase region. Since ErbB3 requires an activating ligand, such as heregulin (NDF), before activated heterodimers can form, molecules that can interfere with the binding of ErbB3 receptor ligands might be used to block or interfere with the formation of ErbB dimers and heterodimers. One example of such a molecule would be a soluble portion of the ectodomain of a receptor molecule that retains tight ligand binding affinity and can therefore “trap” ligands and effectively reduce their concentration so that they cannot activate the ErbB3 receptor.

Several therapeutics exist that attempt to capitalize on this trapping or “decoy” phenomenon. For example, Enbrel™ (etanercept—Amgen) is a soluble, modified version of the TNFR receptor that binds and traps the pro-inflammatory ligand TNFα. In addition, a soluble fusion protein of the VEGFR1 and VEGFR2 receptors, called the VEGF Trap, is currently in clinical trials for the treatment of both macular degeneration and several forms of cancer (Regeneron Pharmaceuticals). An ErbB3 trap has also shown potency in vitro at enhancing the effects of a dual EGFR/ErbB2 inhibitor and reversed GW2974 (a small molecule inhibitor of ErbB1 and ErbB2) resistance in cells treated with NDF.

All currently approved ErbB inhibitors target either EGFR or ErbB2 or both. However, no currently approved therapy interferes with the binding of ligands to multiple ErbB receptors simultaneously. Clearly, new binding molecules are needed that can be used to sequester receptor ligands, such as ErbB ligands, and thereby block ligand binding to multiple ErbB receptors and subsequent receptor activation. Binding molecules capable of binding all known ErbB ligands would be particularly useful. Ideally, if such a molecule could be made it would be a single covalently joined molecule such that only a single molecule. Such a molecule would simplify manufacturing and administration protocols and would theoretically provide maximum benefit when used to sequester receptor ligands. Such molecules will provide excellent therapeutic efficacy, particularly with tumors that overexpress ErbB ligands such as TGFα and NDF.

### SUMMARY

New bivalent ErbB receptor-based ligand binding molecules are disclosed along with their method of preparation and use. The binding molecules are proteins expressed from recombinant DNA molecules. The protein can contain two ErbB extracellular domains that bind ErbB activating ligands. These binding domains act as traps to bind and sequester ligands, thus making them unavailable for binding to cellular ErbB receptors. It has surprisingly been found that portions of the ectodomain of ErbB receptors can be covalently joined together in a single polypeptide such that both binding moieties retain substantial affinity for their respective ligands, such that they can be used to bind and trap ErbB ligands as evidenced by binding in any of a variety of binding assays including, ELISA assays, assays carried out on a Biacore apparatus and the like.

The disclosed proteins can include portions of several ErbB receptors and preferably will bind a wide variety or all known ErbB ligands.

Methods for treating diseases or conditions with the disclosed molecules are also described. Any disease that can be improved, ameliorated, or inhibited by removal or inhibition of an ErbB ligands can be treated by the disclosed methods. The method generally involves preventing the binding of ErbB ligands to the receptors by trapping them in the disclosed binding molecules. In a method. this can be accomplished by administering to a subject in need of treatment a bivalent binding molecule disclosed herein.

Additional features and advantages are described herein, and will be apparent from, the following Detailed Description and the figures.

### BRIEF DESCRIPTION OF THE FIGURES

FIG. 1: Illustrates the ErbB Single Trap mechanism of action and next generation of ErbB Double Trap.

FIG. 2: Illustrates the enhancement of GW2974 cytotoxicity when used with an ErbB single trap therapeutic.

FIG. 3: Provides a photograph of a Western blot of lysates prepared from 293T cells that express the following constructs: 1. pEF-ECD3-IRES-P (single trap containing a portion of the ectodomain of the ErbB3 receptor), 2. pEF-IRES-P (negative control vector), 3. pEF-ECD13-IRES-P 9 (double trap containing a portion of the ectodomain of the ErbB1 receptor on the amino terminal side and ErbB3 receptor on the carboxy terminal side of the polypeptide), 4. pEF-ECD31-IRES-P (double trap containing a portion of the ectodomain of the ErbB3 on the amino terminal side and ErbB1 receptor on the carboxy terminal side of the polypeptide), 5. pEF-ECD14-IRES-P (double trap containing a portion of the ectodomain of the ErbB1 receptor on the amino terminal side and ErbB4 receptor on the carboxy terminal side of the polypeptide), 6. pEF-ECD41-IRES-P (double trap containing a portion of the ectodomain of the ErbB4 receptor on the amino terminal side and ErbB1 receptor on the carboxy terminal side of the polypeptide) and 7. MDA-MB-468 cells (positive antibody control). Constructs were prepared as described in Example 1. The blot was probed with an antibody that recognizes an epitope in the extracellular domain of ErbB1.

FIG. 4: Medium from 293T cells that express the various trap constructs was collected after 3 days. The medium was diluted 1:1000 and ELISA was performed on each sample in duplicate using the Human EGF R DuoSet from R&D Systems. The ELISA assay was read using a Bio-Tek EL312e. The constructs are as follows: VC—Vector control, Her3*—single ErbB3 trap, ECD1-3.p6, ECD3-1.p6, ECD1-4.p5 and ECD4-1.p5. Constructs were prepared as described in Example 1.

FIG. 5: To test the functionality of the traps, conditioned medium from the 293T cells was collected, filtered and diluted 1:1 with fresh medium. This diluted, conditioned medium was then used to culture BT474 cells. After 48 hrs, the cells were fixed and stained with a solution of 1% methylene blue in 50% methanol. BT474 cells were cultured in medium from the trap constructs as follow: 1. pEF-IRES-P (control), 2. pEF-ECD13-IRES-P, 3. pEF-ECD14-IRES-P, 4. pEF-ECD3-IRES-P (single trap), 5. pEF-ECD31-IRES-P and 6. pEF-ECD41-IRES-P abbreviations for constructs are defined above in FIG. 3.

FIG. 6: Cross-linking of hot EGF to traps. Bivalent and monovalent traps were incubated with hot EGF, and with/without excess unlabeled EGF or NDF, followed by cross-linking molecule BS3. Bands shown are either the bivalent or monovalent traps cross-linked to hot EGF. As expected, all bivalent traps bind EGF, while only the ErbB1 monovalent trap binds EGF. Addition of cold EGF competes away the binding of hot EGF in all traps as expected. Interestingly, addition of cold NDF seemed to interfere with the binding of EGF in the ErbB1-ErbB3 bivalent trap but not in the ErbB3-Erb1 or ErbB4-ErbB1 bivalent traps. Constructs were prepared as described in Example 1.

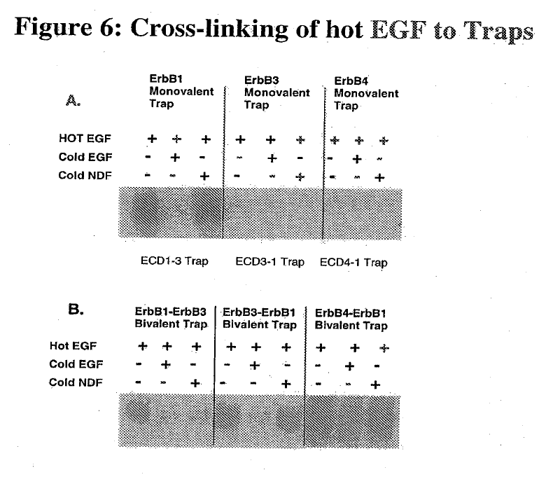

FIG. 7: Binding affinities of the Traps to particular ligands measured by Biacore. Binding affinities (Kd) of the bivalent and monovalent traps for several different ligands were determined using Biacore using standard methods. The traps were bound to Biacore chips and increasing concentrations of ligands were added to determine the binding affinities between the traps and ligands. All bivalent traps could bind both ErbB1 and ErbB3/ErbB4 specific ligands, while the monovalent traps could only bind their respective class of ligands. The full length ectodomain is known to have an affinity for TGFα of about 412-961 nM.

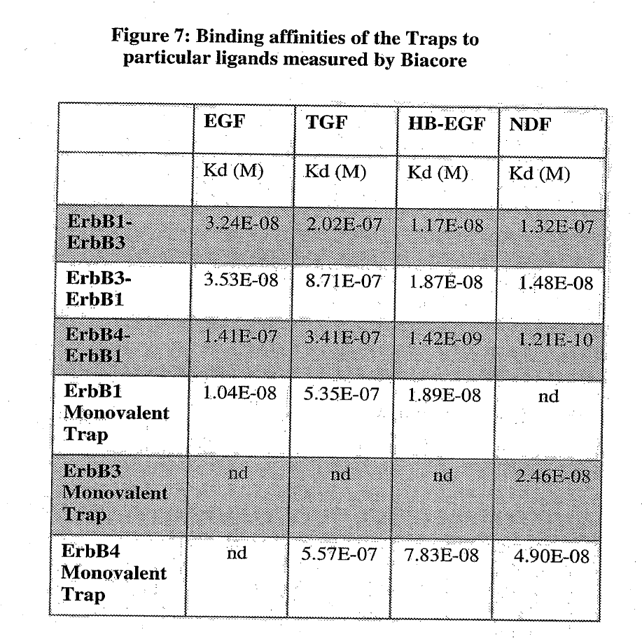

FIG. 8: Binding of labeled EGF (1.6 ng/ml) to EGFR in the presence of traps. EGFR was bound to a Biacore chip and hot EGF was then added. Binding of hot EGF to EGFR in the absence of traps was set at 1. Three different concentrations of both bivalent and monovalent traps were then added to the hot EGF pool before being exposed to the EGFR bound chip. The bivalent traps were able to reduce the pool of hot EGF available while the monovalent ErbB1 trap was not able to at the same concentration.

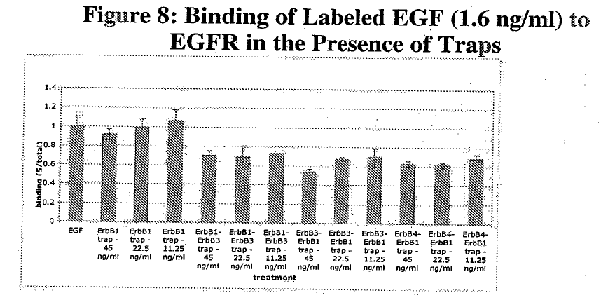

### DETAILED DESCRIPTION

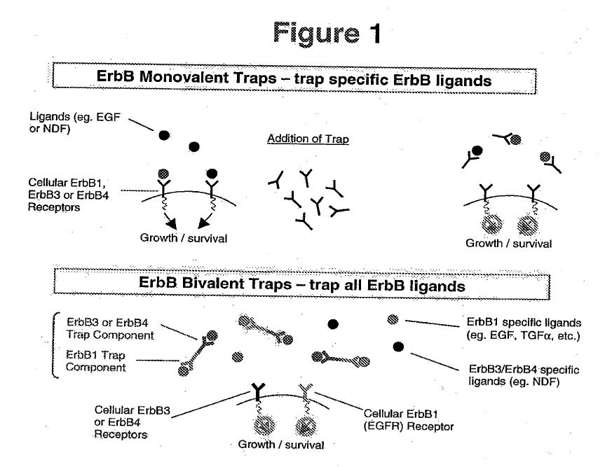

Covalently linked bivalent binding molecules capable of binding ligands to multiple receptors, such as ErbB receptors are disclosed. Preferred bivalent binding molecules are capable of binding ligands for at least two distinct receptors. Such binding molecules are termed “double traps” for purposes of this specification. In one embodiment, the molecules have substantial affinity for all ErbB ligands. Exemplary embodiments of binding molecules are illustrated diagrammatically in FIG. 1. FIG. 1 also illustrates the mechanism by which such dual binding molecules are thought to operate.

In an embodiment, the invention relates to bivalent binding molecules having substantial binding affinity for ligands that bind distinct receptors. The bivalent binding molecules can include portions of the ectodomains of receptors and are preferably covalently joined in a single polypeptide sequence. In instances where the spectrum of ligands bound by two receptors overlap, each binding moiety of the bivalent binding molecule made from portions of those receptors may bind similar or identical ligands. It is preferred that the bivalent binding molecule be soluble in aqueous solutions.

In an embodiment, each binding moiety of the bivalent binding molecule can be a soluble portion containing extracellular domain of a receptor. Any suitable receptor can be utilized in the binding molecule. Suitable receptors will generally contain extracellular or intracellular domains that contain all of the determinants necessary and sufficient for ligand binding. In an embodiment, various members of the family of ErbB receptors can be used to create bivalent binding molecules. Thus, the bivalent binding molecule can be a combination of the extracellular ligand binding domains of ErbB receptors, for example ErbB1 and ErbB3, ErbB1 and ErbB4 or other combinations. The binding domains can exist in any order on the polypeptide chain so long as suitable binding affinity for receptor ligands is maintained.

For purposes of this application suitable binding affinities are affinities that are high enough to trap ErbB ligands in a physiological matrix. Preferably, dissociation constants will be no higher than about 100-fold to about 1,000-fold above the dissociation constants of the native receptors. More preferably, dissociation constants in the nanomolar range or lower are preferred. Nevertheless, any affinity that is sufficient to bind and trap ErbB ligands thereby preventing or interfering with their binding to ErbB receptors are suitable for use in the disclosed compositions and can find use in the disclosed methods.

The complete nucleotide sequences of the ErbB1, ErbB2, ErbB3 and ErbB4 are known and can be found in Genbank as accession #: NM_{—}005228 for ErbB1, accession # NM_{—}004448 for ErbB2, accession #: M29366 or NM_{—}001982 for ErbB3, and accession #: NM_{—}005235 for ErbB4. For purposes of this specification, a full length EGFR ectodomain refers to the ectodomain consisting of amino acid residues 1-621 of ErbB1 or equivalent residues of other members of the EGF receptor family. The amino acid sequence of the full length ectodomains for the ErbB receptor family is also known, portions of these sequences are included below as SEQ ID NO. 2 for ErbB1 amino acid residues 1-532, SEQ ID NO. 22 for ErbB1 amino acid residues 1-500, SEQ ID NO 6 for ErbB3 amino acid residues 1-531, SEQ ID NO. 24 for ErbB3 amino acid residues 1-499, SEQ ID NO 8 for ErbB4 amino acid residues 1-528, and SEQ ID NO. 26 for ErbB4 amino acid 1-496. In each sequence position number “1” is the first amino acid following the signal peptide. Corresponding nucleotide sequences that encode these amino acids can be found as SEQ ID NOS. 2, 6 and 8, respectively. The full length ectodomain for ErbB receptors contains four sub-domains, referred to as L1, CR1, L2 and CR2, where L and CR are acronyms for large and cys-rich respectively. Amino acid sequence alignments of the ectodomains of ErbB1, ErbB2, ErbB3 and ErbB4 have been determined. See US Patent Publication No. 2006/0234343, FIGS. 1A and 1B.

The CR2 sub-domain of ErbB receptors is thought to link the ligand binding domain (L1, CR1 and L2) with the membrane spanning region and consists of seven additional modules which are joined by linkers of 2 or 3 amino acid residues and bounded by cysteine residues. For ErbB1 these modules extend from amino acid positions 482-499, 502-511, 515-531, 534-555, 558-567, 571-593, and 596-612 for modules 1-7, respectively. For ErbB2 these modules extend from 490-507, 510-519, 523-539, 542-563, 566-575, 579-602 and 605-621 for modules 1-7, respectively. For ErbB3 481-498, 501-510, 514-530, 533-554, 557-566, 570-591, and 594-610 for modules 1-7, respectively. For ErbB4 these modules extend from 478-495, 498-507, 511-527, 530-552, 555-564, 568-589, and 592-608 for modules 1-7, respectively.

Suitable portions of ErbB ectodomains can be prepared by any suitable recombinant DNA technology, as is known in the art and described herein in the examples. For example nucleotide sequences encoding the desired ectodomains or portions of ectodomains can now be custom manufactured, ligated together and cloned into expression vectors. The expression vectors can then be used to transform cells which express the protein and the binding molecules can then be purified from the cells or a cell supernatant. The ectodomains can include the full length ectodomain of each receptor. Alternatively, the ectodomains can be truncated at either the amino or carboxy terminal ends. At the amino-terminal end, the ectodomains can begin at position 1, 2, 3, 4, 5, 6, 7, 8, 9, 10 or more, for example, so long as the binding activity of the resulting binding moiety is not substantially diminished. At the carboxy-terminal end, the ectodomains can terminate after or within the seventh module, sixth module, fifth module, fourth module, third module, second module, first module or even before the first module, for example with reference to ErbB1, at amino acid number 500, 512, 532, 556, 568, 594, 613 and at corresponding positions for ErbB3 and ErbB4. Thus, for ErbB1 amino acids 1-532 [SEQ ID NO 2] or 1-500 [SEQ ID NO 22] can be used, for example. For ErbB3 amino acids 1-499 [SEQ ID NO. 24] or 1-531 [SEQ ID NO 6] can be used, among other sequences. For ErbB4 amino acids 1-496 [SEQ ID NO 26] or 1-528 [SEQ ID NO 8] can be used, among others.

In an embodiment, the amino acid sequence of one or both of the binding moieties may be modified provided that the modification does not adversely affect the binding affinity of the binding moiety for its ligand(s). For example, modified binding moieties may be constructed by making various substitutions of residues or sequences or deleting terminal or internal residues or sequences not needed for binding activity. Generally, substitutions should be made conservatively; for example, the most preferred substitute amino acids are those having physiochemical characteristics resembling those of the residue to be replaced. Similarly, when a deletion or insertion strategy is adopted, the potential effect of the deletion or insertion on biological activity should be considered. In order to preserve the biological activity of the binding moieties, deletions and substitutions will preferably result in homologous or conservatively substituted sequences, meaning that a given residue is replaced by a biologically similar residue. Examples of conservative substitutions include substitution of one aliphatic residue for another, such as Ile, Val, Leu, Met or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Moreover, particular amino acid differences between human, murine or other mammalian EGFRs is suggestive of additional conservative substitutions that may be made in ErbB binding moieties without altering the essential biological characteristics of the binding moiety.

In an embodiment, bivalent binding molecules can be arranged in the following motifs: B-L-B-F; B-L-rB-F and B-F-B. B represents a binding moiety which can originate from a receptor. The binding moieties can be the same or different. rB represents a binding moiety in which the amino acid sequence is reversed such that the amino-terminal amino acids become the carboxy-terminal residues. An exemplary sequence for ErbB1 is SEQ ID NO 3 which is a nucleotide sequence encoding one such reverse sequence of SEQ ID NO. 1 to provide an amino sequence of SEQ ID NO. 4 which is the reverse of the sequence in SEQ. ID NO. 2. Similar inversions can be constructed for ErbB3 and ErbB4, as desired. Such reverse sequences can be positioned as the carboxy-terminal binding moiety to mimic the structure of receptors as they are found in the membrane.

In preferred embodiments, the two binding moieties are different. Suitable arrangements include, for example, B1-L-B2-F, B2-L-B1-F, B1-F-B2, B2-F-B1. In particular embodiments, B1 and B2 are different and are portions of the ectodomain of ErbB1, ErbB3 and ErbB4. In one particularly preferred embodiment B1 and B2 are ErbB1 and ErbB4, respectively. More specifically, with respect to ErbB1, amino acids 1-500 and 1-532 can be used to form an active binding molecule and with respect to ErbB4 amino acids 1-496 and 1-528 can be used such that when ErbB1 and ErbB4 are joined in a single polypeptide they form a bivalent binding molecule having a substantial affinity for both ErbB1 and ErbB4 ligands regardless of whether ErbB1 is positioned on the amino or carboxy-terminal side of ErbB4. Of course, B1 or B2 could be any other receptor or ligand binding protein and may not necessarily begin with amino acid number one.

“L” is an optional linker moiety which can be used to join binding moieties. Many suitable linker molecules are known and can be used. Preferably, the linker will be non-immunogenic. For linkers and methods of identifying desirable linkers, see, for example, George et al. (2003) Protein Engineering 15:871-879, herein specifically incorporated by reference. A linker sequence may include one or more amino acids naturally connected to a binding moiety and can be added to provide specifically desired sites of interest, allow component domains to form optimal tertiary structures and/or to enhance the interaction of a component with its target molecule. One simple linker is (Gly₄Ser)_{X} wherein “X” can be any number from 1 to about 10 or more in certain embodiment linkers wherein “X” is three [SEQ ID NO: 29] have found use. However, the linker can also be an amide bond.

“F” is an optional fusion partner and can be any component that enhances the functionality of the bivalent binding molecule. Suitable fusion partners may enhance the biological activity of the bivalent binding molecule, aid in its production and/or recovery, or enhance a pharmacological property or the pharmacokinetic profile of the fusion polypeptide by, for example, enhancing its serum half-life, tissue penetrability, lack of immungenicity, or stability.

When the fusion partner is a serum protein or fragment thereof, it can be (α-1-microglobulin, AGP-1, orosomuciod, α-acid glycoprotein, vitamin D binding protein (DBP), hemopexin, human serum albumin (hSA), transferrin, ferritin, afamin, haptoglobin, α-fetoprotein thyroglobulin, α-2-HS-glycoprotein, β-2-glycoprotein, hyaluronan-binding protein, syntaxin, C1R, C1q a chain, galectin3-Mac2 binding protein, fibrinogen, polymeric Ig receptor (PIGR), (α-2-macroglobulin, urea transport protein, haptoglobin, IGFBPs, macrophage scavenger receptors, fibronectin, giantin, Fc (especially including an IgG Fc domain), α-1-antichyromotrypsin, α-1-antitrypsin, antithrombin III, apolipoprotein A-I, apolipoprotein B, β-2-microglobulin, ceruloplasmin, complement component C3 or C4, CI esterase inhibitor, C-reactive protein, cystatin C, and protein C. The inclusion of a fusion partner component may extend the serum half-life of the fusion polypeptide of the invention when desired.

For the ErbB receptors, known ligands and receptor binding specificity is shown below in Table I. Thus, combination of an ErbB1 and ErbB3 binding moiety can be used to create a bivalent binding molecule with specificity for EGF, TGFα, HB-EGF, Betacellulin, Amphiregulin, Epiregulin, Epigen, Neuregulin 1α, Neuregulin 1β, Neuregulin 2α and Neuregulin 2 β. The combination of binding domains for ErbB1 and ErbB4 have binding affinity for EGF, TGFα, HB-EGF, Betacellulin, Amphiregulin, Epiregulin, Epigen, Neuregulin 1α, Neuregulin 1β, Neuregulin 2α, Neuregulin 2β, Neuregulin 3 and Neuregulin 4, which includes all of the known ErbB ligands.

TABLE I
Ligand Receptor Specificity

| | |
|---|---|
| ErbB1 | EGF |
| | TGFα |
| | HB-EGF |
| | Betacellulin |
| | Amphiregulin |
| | Epiregulin |
| | Epigen |
| ErbB3 | Neuregulin 1α |
| | Neuregulin 1β |
| | Neuregulin 2α |
| | Neuregulin 2β |
| ErbB4 | Betacellulin |
| | HB-EGF |
| | Epiregulin |
| | Neuregulin 1α |
| | Neuregulin 1β |
| | Neuregulin 2α |
| | Neuregulin 2β |
| | Neuregulin 3 |
| | Neuregulin 4 |

Bivalent binding molecules will also generally include signal sequences at their amino terminal ends. Any suitable signal sequence, of which many are known, can be used. For example, the ErbB ectodomain in the first position of the bivalent binding molecule can contain its own native signal peptide. Alternatively, that signal peptide can be modified to conform to a consensus Kozak sequence (GCCGCCACCATGG) where ATG is the start codon of the ErbB ectodomain and the position at +4 is changed to G to conform to a consensus Kozak sequence. Suitable sequences can be found in Table 2 below.

TABLE 2
Signal Peptide Sequences

Suitable ErbB1 signal peptide:
Normal nucleotide sequence

[SEQ ID NO. 9]

ATGCGACCCTCCGGGACGGCCGGGGCAGCGCTCCTGGCGCTGCTGGCTGC
GCTCTGCCCGGCGAGTCGGGCT
Normal amino acid sequence

[SEQ ID NO. 10]

M R P S G T A G A A L L A L L A A L C P A S R A
Modified nucleotide sequence

[SEQ ID NO. 11]

ATGGGACCCTCCGGGACGGCCGGGGCAGCGCTCCTGGCGCTGCTGGCTGC
GCTCTGCCCGGCGAGTCGGGCT
Modified amino acid sequence

[SEQ ID NO 12]

M G P S G T A G A A L L A L L A A L C P A S R A
Suitable ErbB3 signal peptide:
Normal nucleotide sequence

[SEQ ID NO 13]

ATGAGGGCGAACGACGCTCTGCAGGTGCTGGGCTTGCTTTTCAGCCTGGC
CCGGGGC
Normal amino acid sequence

[SEQ ID NO 14]

M R A N D A L Q V L G L L F S L A R G
Modified nucleotide sequence

[SEQ ID NO 15]

ATGGGGGCGAACGACGCTCTGCAGGTGCTGGGCTTGCTTTTCAGCCTGGC
CCGGGGC
Modified amino acid sequence

[SEQ ID NO 16]

M G A N D A L Q V L G L L F S L A R G
Suitable ErbB4 signal peptide
Normal nucleotide sequence

[SEQ ID NO 17]

ATGAAGCCGGCGACAGGACTTTGGGTCTGGGTGAGCCTTCTCGTGGCGGC
GGGGACCGTCCAGCCCAGCGATTCT
Normal amino acid sequence

[SEQ ID NO 18]

M K P A T G L W V W V S L L V A A G T V Q P S D S
Modificd nucleotide sequence

[SEQ ID NO 19]

ATGGGGCCGGCGACAGGACTTTGGGTCTGGGTGAGCCTTCTCGTGGCGGC
GGGGACCGTCCAGCCCAGCGATTCT
Modified amino acid sequence

[SEQ ID NO 20]

M G P A T G L W V W V S L L V A A G T V Q P S D S

The disclosed bivalent binding molecules will include amino acid sequences expressed from recombinant DNA molecules. As indicated above, the recombinant DNA molecule can include a first nucleotide sequence encoding a portion of a first receptor protein and a second nucleotide sequence encoding a portion of a second receptor protein. The receptor proteins can be the same or different, however it is generally preferred to include different receptor proteins so that the bivalent binding molecule will bind a broader spectrum of binding molecules. In such cases the first and second receptor proteins are generally encoded from different genes.

Nucleotide sequences that encode the bivalent binding moieties, optional linker and an optional fusion partner can be cloned into a recombinant DNA construct in an arrangement with transcription and translation sequences such that the bivalent binding molecule can be expressed as a single polypeptide chain in a suitable host. Any of the methods known to one skilled in the art for the insertion of DNA fragments into a vector may be used to construct expression vectors encoding the fusion polypeptides of the invention under control of transcriptional/translational control signals. It is well within the skill of one having skill in the art to select transcription and translation sequences that can be used to express genes in suitable hosts. Any host cell that can produce the disclosed molecules from their recombinant genes can be used. Suitable host cells include, but are not limited to, bacterial, yeast, insect, and mammalian cells. In many circumstances receptors are glycosylated and glycosylation can influence ligand binding. Thus, the selection of a host can depend on the glycosylation pattern generated by the host cell. Any host cell that can produce ligand binding molecules with suitable binding affinities can be used. In the case of an ErbB-containing binding molecule a mammalian host cell can be used for example and, more specifically CHO cells, for example.

Many suitable promoter and enhancer elements are known in the art. Promoters that may be used to control expression of the chimeric polypeptide molecules include, but are not limited to, a long terminal repeats; SV40 early promoter region, CMV, M-MuLV, thymidine kinase promoter, the regulatory sequences of the metallothionine gene; prokaryotic expression vectors such as the β-lactamase promoter, or the tac promoter; promoter elements from yeast or other fungi such as Gal 4 promoter, ADH, PGK, alkaline phosphatase, and tissue-specific transcriptional control regions derived from genes such as elastase I.

The disclosed bivalent binding molecules may be purified by any technique which allows for stable bivalent binding of the resulting double trap molecules. For example, the bivalent binding molecules may be recovered from cells either as soluble proteins or as inclusion bodies, from which they may be extracted quantitatively by 8M guanidinium hydrochloride and dialysis, as is known. Alternatively, the bivalent binding molecules, conventional ion exchange chromatography, hydrophobic interaction chromatography, reverse phase chromatography or gel filtration may be used. Affinity techniques that utilize immobilized ligands or ligand mimetics can also be used.

Binding affinity and inhibitor potency of the bivalent binding molecules can be measured for candidate truncated ectodomains using biosensor technology or by classic binding assays such as ELISA which are well known in the art.

The bivalent binding molecules can be used as a monotherapy or in combination therapies. In numerous embodiments, a bivalent binding molecule may be administered in combination with one or more additional compounds or therapies, including a chemotherapeutic agent, surgery, catheter devices, and radiation. Combination therapy includes administration of a single pharmaceutical dosage formulation which contains a bivalent binding molecule and one or more additional agents; as well as administration of a bivalent binding molecule and one or more additional agent(s) in its own separate pharmaceutical dosage formulation. For example, a bivalent binding molecule and a cytotoxic agent, a chemotherapeutic agent or a growth inhibitory agent can be administered to the patient together in a single dosage composition such as a combined formulation, or each agent can be administered in a separate dosage formulation. More specifically, the bivalent binding molecules can be used in combination therapies with therapeutic agents such as Lapatinib, Herceptin™, Erbitux and the like. Where separate dosage formulations are used, the fusion polypeptide of the invention and one or more additional agents can be administered concurrently, or at separately staggered times, i.e., sequentially.

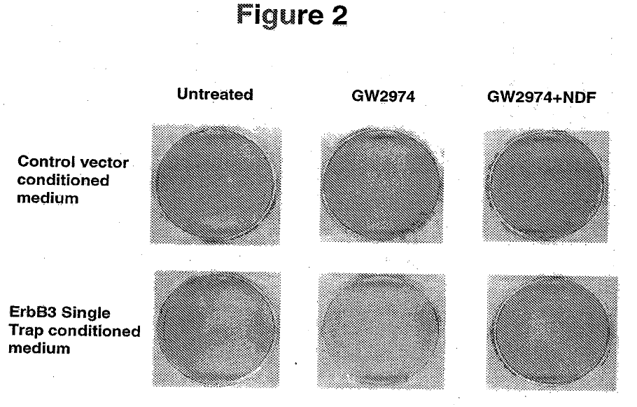

FIG. 2 demonstrates the in vitro efficacy of several bivalent binding molecules when tested with breast cancer cell cultures. In the top row of FIG. 2 breast cancer cells were cultured in either control medium (top row) or medium previously conditioned with the ErbB3 ligand binding molecule “single trap” or univalent binding molecule (bottom row). The cells were then either untreated, treated with 1 μM GW2974 (generic GW572016) or with GW2974+NDF (heregulin). As can be seen, the ErbB3 single trap enhanced the dual inhibitor toxicity and reversed the NDF dependent resistance to the dual inhibitor.

The present invention also provides pharmaceutical compositions comprising a bivalent binding molecule of the invention. Such compositions comprise a therapeutically effective amount of a bivalent binding molecule, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Pharmaceutically acceptable carriers include other ingredients for use in formulations such as DPPC, DOPE, DSPC and DOPC. Natural or synthetic surfactants may be used. PEG may be used (even apart from its use in derivatizing the protein or analog), Dextrans, such as cyclodextran, may be used. Bile salts and other related enhancers may be used. Cellulose and cellulose derivatives may be used. Amino acids may be used, such as use in a buffer formulation. Pharmaceutically acceptable diluents include buffers having various contents (e.g., Tris-HCl, acetate, phosphate), pH and ionic strength; additives such as detergents and solubilizing agents (e.g., TWEED™80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations. Liposome, microcapsule or microsphere, inclusion complexes, or other types of carriers are also contemplated.

The amount of the active bivalent binding molecule that will be effective for its intended therapeutic use can be determined by standard clinical techniques based on the present description. In addition, in vitro assays may optionally be employed to help identify optimal dosage ranges. Generally, the daily regimen should be in the range of 0.1-1000 micrograms of the active per kilogram of body weight, preferably 0.1-150 micrograms per kilogram. Effective doses may be extrapolated from dose-response curves derived from in vitro or animal model test systems. Dosage amount and interval may be adjusted individually to provide plasma levels of the compounds that are sufficient to maintain therapeutic effect. In cases of local administration or selective uptake, the effective local concentration of the compounds may not be related to plasma concentration. The dosage regimen involved in a method for treatment will be determined by the attending physician, considering various factors which modify the action of drugs, e.g. the age, condition, body weight, sex and diet of the patient, the severity of disease, time of administration and other clinical factors.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

A method for treating a patient in need of treatment is disclosed that includes obtaining a binding molecule that binds an ErbB ligand and removing a portion of the ligand from the serum. A binding molecule as disclosed herein can be immobilized to a solid support such as an apheresis or biocore support by standard methods. When the binding molecule is immobilized to a solid support the serum or blood of the patient can be placed in contact with the solid support in the apheresis column to remove a portion of the ErbB ligand from the blood.

In a method the disclosed bivalent binding molecules can be used in diagnostic methods for the detection of over expression of ErbB ligands. A cancer characterized by excessive activation of an ErbB receptor can be caused by excessive activation over that in non-cancerous cells of the same tissue type. Such excessive activation can be caused by overexpression of the ErbB receptor and/or greater than normal levels of an ErbB ligand.

In an embodiment, a cancer can be subjected to a diagnostic or prognostic assay to determine whether excessive activation of the ErbB receptor is caused by over expression of an ErbB ligand. The bivalent binding molecules can be labeled with any detectable marker such as radioactivity or contrast markers. The molecules can then be contacted with cancer cells and visualized using standard methods known in the art. For example, the method can be carried out by administering a bivalent binding molecule which binds the molecule to be detected and is tagged with a detectable label (e.g. a radioactive isotope) and externally scanning the patient for localization of the label.

### Example 1

The present example demonstrates the construction of representative compositions of bivalent binding molecules having two ErbB receptor extracellular domains.

The ErbB bivalent binding molecules were designed to bind to all ligands of the ErbB family by incorporating the extracellular domains of ErbB1 and ErbB3 or ErbB1 and ErbB4. Two different orientations were designed for each pair. Thus the following combinations were prepared: ErbB1-ErbB3, ErbB3-ErbB1, ErbB1-ErbB4 and ErbB4-ErbB1.

The pcDNA3.1(+) vector was used as a cloning vehicle to facilitate construction of the constructs because of its extensive multiple cloning site. First, oligonucleotides for a tobacco etch virus (TEV) protease recognition sequence (ETVRFQG/S) [SEQ ID NO: 27] followed by a 6× histidine tag and stop codon were cloned into the XbaI and ApaI sites of pcDNA3.1(+) to yield pcDNA3.1 (+)-TH. The oligonucleotides included a NotI site upstream of the ApaI site so the construct could eventually be liberated from pcDNA3.1(+). The TEV-6×His-STOP sense oligonucleotide having with an XbaI site and NotI site embedded upstream of the ApaI site: 5′ CTA GAG AAA ACC TGT ACT TCC AGT CCC ATC ATC ATC ATC ATC ATT GAG CGG CCG CGG GCC [SEQ ID NO 28] was used along with the TEV-6×His-STOP anti-sense oligonucleotide with an XbaI site and NotI site embedded upstream of the ApaI site: 5′ CGC GGC CGC TCA ATG ATG ATG ATG ATG ATG GGA CTG GAA GTA CAG GTT TTC T [SEQ ID NO 30].

The first 3 subdomains (LI, SI, LII as are known) and the 1^{st} module of the 4^{th} subdomain (SII as is known) of the extracellular domain of either ErbB1 or ErbB4 were cloned into the NheI and KpnI sites of pcDNA3.1(+)-TH, along with a linker sequence. Specifically, the linker sequence encodes a 15 amino acid (Gly₄Ser)₃ peptide composed of: Gly-Gly-Gly-Gly-Ser-Gly-Gly-Gly-Gly-Ser-Gly-Gly-Gly-Gly-Ser [SEQ ID NO: 29]. Forward PCR primers were designed to amplify the signal peptide plus the LI, SI, LII and 1^{st} module of the SII subdomains of both ErbB1 and ErbB4. A consensus “Kozak” sequence, as is known, was incorporated into the primers immediately upstream of the signal peptide start codon. Reverse PCR primers were designed to include up to and including the V500 amino acid of ErbB1 and the L496 amino acid of ErbB4 (the leucine after the signal peptide of ErbB1 is defined as L1 amino acid and the glutamine after the signal peptide of ErbB4 is defined as Q1 amino acid) followed by an AgeI site, the (Gly₄Ser)₃ linker sequence [SEQ ID NO 29] and a KpnI site. The ErbB1 forward primer sequence with NheI site and consensus “Kozak” sequence was as follows: 5′ AGC TGC TAG CGC CAC CAT GCG ACC CTC CGG GAC GGC CG [SEQ ID NO 31]. The ErbB4 forward primer sequence with NheI site and consensus “Kozak” sequence was as follows: 5′ AGC TGC TAG CGC CAC CAT GAA GCC GGC GAC AGG ACT TT [SEQ ID NO 32]. The ErbB1 reverse primer sequence with AgeI site, (Gly₄Ser)₃ linker sequence and KpnI site was 5′ TCT GGT ACC CGA TCC GCC ACC GCC AGA GCC ACC TCC GCC TGA ACC GCC TCC ACC ACC GGT GAC GCA GTC CCT GGG CTC CGG GCC C [SEQ ID NO 33]. The ErbB1 reverse primer sequence with AgeI site, (Gly₄Ser)₃ [SEQ ID NO: 29] linker sequence and KpnI site is 5′ TCT GGT ACC CGA TCC GCC ACC GCC AGA GCC ACC TCC GCC TGA ACC GCC TCC ACC ACC GGT CAG ACA TTG GTC TGG CCC AGG TCC C [SEQ ID NO 34].

The extracellular domains were amplified from full-length cDNAs of either ErbB1 or ErbB4. This yielded the plasmids pcDNA3.1(+)-ECD1-GS-TH and pcDNA3.1 (+)-ECD4-GS-TH.

To construct ErbB3 constructs, the first 3 subdomains (L1, SI and LII) and the 1^{st} module of the 4^{th} subdomain (SII) of the extracellular domain of ErbB3 were amplified by PCR from a full-length cDNA and cloned into the NheI and AgeI sites of pcDNA3.1(+)-ECD4-GS-TH. The forward PCR primer was designed, as previously described, to incorporate a NheI site and a consensus “Kozak” sequence immediately upstream of the signal peptide start codon of ErbB3. The ErbB3 forward primer sequence with NheI site and consensus “Kozak” sequence was 5′ AGC GCT AGC GCC ACC ATG AGG GCG AAC GAC GCT CTG CAG G [SEQ ID NO 35]. The ErbB3 reverse primer sequence with AgeI site was 5′ AGC ACC GGT CAA GCA CTG ACC AGG GCC TGG GCC C [SEQ ID NO 36]

The extracellular domain was amplified from a full-length cDNA of ErbB3 and used to produce the plasmid pcDNA3.1(+)-ECD3-GS-TH.

The second ErbB extracellular domain was cloned into each construct. This was done by amplifying the first 3 subdomains (L1, SI and LII) and the 1^{st} module of the SII subdomain of the extracellular domain of either ErbB1, ErbB3 or ErbB4. The only difference between the extracellular domains placed in the second position of the construct, as compared with the first position, was that the signal peptide was not included. Forward PCR primers with a KpnI site were designed to amplify the first 3 subdomains and 1^{st} module of the 4^{th} subdomain of either ErbB1, ErbB3 or ErbB4. Reverse PCR primers with an XbaI site were also designed to either ErbB 1, ErbB3 or ErbB4. The last amino acid for each extracellular domain was V500 (ErbB1), L499 (ErbB3) and L496 (ErbB4). The ErbB1 second position forward primer with KpnI site was 5′ CGG GGT ACC CTG GAG GAA AAG AAA GTT TGC C [SEQ ID NO 37]. The ErbB3 second position forward primer with KpnI site was 5′ CGG GGT ACC TCC GAG GTG GGC AAC TCT CAG GCA G [SEQ ID NO.: 38]. The ErbB4 second position forward primer with KpnI site was 5′ CGG GGT ACC CAG TCA GTG TGT GCA GGA ACG G [SEQ ID NO.: 39]. The ErbB1 second position reverse primer with XbaI site was 5′ TGC TCT AGA GAC GCA GTC CCT GGG CTC CGG G [SEQ ID NO.: 40]. The ErbB3 second position reverse primer with XbaI site was 5′ TGC TCT AGA CAA GCA CTG ACC AGG GCC TGG GCC C [SEQ ID NO.: 41]. The ErbB4 second position reverse primer with XbaI site was 5′ TGC TCT AGA CAG ACA TTG GTC TGG CCC AGG T [SEQ ID NO.: 42].

The extracellular domain of ErbB1 was cloned into the second position in both pcDNA3.1(+)-ECD3-GS-TH and pcDNA3.1(+)-ECD4-GS-TH to yield the plasmids pcDNA3.1 (+)-ECD3-GS-ECD1-TH and pcDNA3.1 (+)-ECD4-GS-ECD1-TH. The extracellular domain of ErbB3 was cloned into the second position of pcDNA3.1 (+)-ECD 1-GS-TH to yield the plasmid pcDNA3.1 (+)-ECD1-GS-ECD3-TH. The extracellular domain of ErbB4 was cloned into the second position of pcDNA3.1 (+)-ECD1-GS-TH to yield the plasmid pcDNA3.1 (+)-ECD1-GS-ECD4-TH.

All constructs were verified by direct sequencing. The bicistronic plasmid, pEF-IRES-P, which contains the Elongation Factor 1 alpha (EF1α) promoter and the puromycin resistance gene expressed from an IRES sequence after the multiple cloning site was used for expression. All 4 double trap constructs were liberated from pcDNA3.1 (+) by restriction endonuclease digestion with NheI and NotI and cloned into the same sites in pEF-IRES-P. This yielded the plasmids pEF-ECD13-IRES-P, pEF-ECD31-IRES-P, pEF-ECD 14-IRES-P and pEF-ECD41-IRES-P, which were used to express the ErbB1-ErbB3, ErbB3-ErbB1, ErbB1-ErbB4, and ErbB4-ErbB1 proteins, respectively, with the first binding moiety being located toward the amino terminus of the protein.

### Example 2

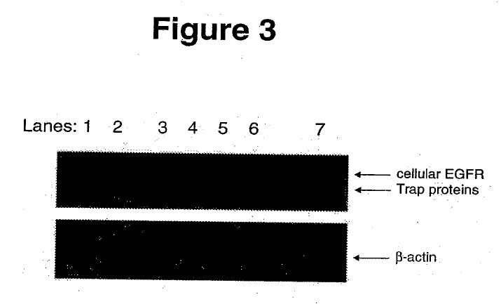

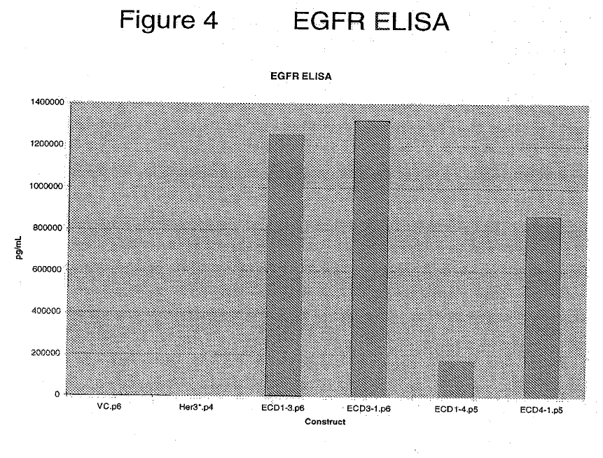

This example demonstrates expression of a double trap molecule from a recombinant DNA molecule in a mammalian host cell and its purification in active form. All 4 constructs from EXAMPLE 1 were digested with PvuI to generate a linear recombinant DNA molecule, which is more suitable for stable integration into the cellular genomic DNA. The 4 linearized double trap molecules were transfected by standard methods into 293T cells, which were then selected in increasingly higher concentrations of puromycin to generate a population of cells with stable integration of the constructs. Expression of the constructs can be assessed by different means such as a western blot to detect levels of the trap in the cells prior to secretion, as shown in FIG. 3, or ELISA, as shown in FIG. 4, to determine the concentration of the binding molecules in the cell culture medium. The ELISA assay was used to screen a large number of individual cells to establish clonally derived cell lines that express the highest levels of the trap molecules. The binding molecules contain a histidine tag which allowed the molecules to be purified by simple affinity purification methods.

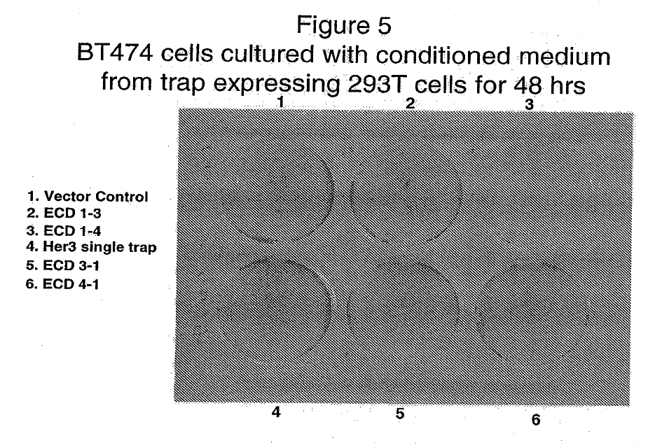

To test the functionality of the binding molecules, conditioned medium from the 293T cells was collected, filtered and used to culture BT474 cells. A significant reduction in cell number was observed after 48 hrs in the BT474 cells cultured with medium from 293T cells that express the pEF-ECD14-IRES-P construct. See FIG. 5

The invention is claimed as follows:

1. A bivalent binding molecule having binding affinity for a first and a second ErbB ligand at separate binding sites in a single covalently joined protein molecule.

2. The binding molecule of claim 1, wherein the binding molecule is soluble in an aqueous solution.

3. The binding molecule of claim 1, wherein the binding molecule further comprises a portion of an extracellular domain of an ErbB receptor that binds a ligand to an ErbB receptor.

4. The binding molecule of claim 1, further comprising a portion of an extracellular domain from ErbB1 that binds a ligand for ErbB1.

5. The binding molecule of claim 1, further comprising a portion of an extracellular domain from ErbB1 that binds a ligand for ErbB1 wherein the portion includes amino acids 1-500 of the ErbB receptor.

6. The binding molecule of claim 1, further comprising a portion of an extracellular domain from ErbB1 that binds a ligand for ErbB1 wherein the portion includes amino acids 1-532 of the ErbB1 receptor.

7. The binding molecule of claim 1, further comprising a portion of an extracellular domain from ErbB1 that binds a ligand for ErbB1 wherein the portion includes amino acids 1-621 of the ErbB1 receptor.

8. The binding molecule of claim 1, further comprising a portion of an extracellular domain from ErbB3 that binds a ligand for ErbB3.

9. The binding molecule of claim 1, further comprising a portion of an extracellular domain from ErbB3 that binds a ligand for ErbB3 wherein the portion includes amino acids 1-499 of the ErbB3 receptor.

10. The binding molecule of claim 1, further comprising a portion of an extracellular domain from ErbB3 that binds a ligand for ErbB3 wherein the portion includes amino acids 1-531 of the ErbB3 receptor.

11. The binding molecule of claim 1, further comprising a portion of an extracellular domain from ErbB3 that binds a ligand for ErbB3 wherein the portion includes amino acids 1-624 of the ErbB3 receptor.

12. The binding molecule of claim 1, further comprising a portion of an extracellular domain from ErbB4 that binds a ligand for ErbB4.

13. The binding molecule of claim 1, further comprising a portion of an extracellular domain from ErbB4 that binds a ligand for ErbB4 wherein the portion includes amino acids 1-496 of the ErbB4 receptor.

14. The binding molecule of claim 1, further comprising a portion of an extracellular domain from ErbB4 that binds a ligand for ErbB4 wherein the portion includes amino acids 1-528 of the ErbB4 receptor.

15. The binding molecule of claim 1, further comprising a portion of an extracellular domain from ErbB4 that binds a ligand for ErbB4 wherein the portion includes amino acids 1-626 of the ErbB4 receptor.

16. The binding molecule of claim 1, further comprising a portion of an extracellular domain from ErbB1 that binds a ligand for ErbB1 and a portion of an extracellular domain from ErbB3 that binds a ligand for ErbB3.

17. The binding molecule of claim 1, further comprising a portion of an extracellular domain from ErbB1 that binds a ligand for ErbB1 and a portion of an extracellular domain from ErbB4 that binds a ligand for ErbB4.

18. The binding molecule of any of claims 16 and 17, wherein the carboxy-terminal ErbB ligand binding site has an amino acid sequence that is reversed in the amino to carboxy terminal direction.

19. The binding molecule of any of claims 1-18, further comprising a linker between the binding sites.

20. The binding molecule of any of claims 1-18, further comprising a fusion partner.

21. A recombinant DNA molecule encoding a protein having binding affinity for a first and a second ErbB ligand at separate binding sites in a single covalently joined protein molecule.

22. The recombinant DNA molecule of claim 21, further comprising a nucleotide sequence encoding a portion of an ErbB receptor protein that binds a ligand for ErbB.

23. The recombinant DNA molecule of claim 21, further comprising a nucleotide sequence encoding a portion of an ErbB receptor protein that binds a ligand for ErbB and a second nucleotide sequence encoding a portion of an ErbB receptor protein that binds a ligand for ErbB.

24. The recombinant DNA molecule of claim 21, further comprising a nucleotide sequence encoding a portion of an ErbB1 receptor protein that binds a ligand for ErbB1.

25. The recombinant DNA molecule of claim 21, further comprising a nucleotide sequence encoding amino acids 1-500 of the ErbB1 receptor.

26. The recombinant DNA molecule of claim 21, further comprising a nucleotide sequence encoding amino acids 1-532 of the ErbB1 receptor.

27. The recombinant DNA molecule of claim 21, further comprising a nucleotide sequence encoding a portion of an extracellular domain from ErbB1 wherein the portion encodes amino acids 1-621 of the ErbB1 receptor.

28. The recombinant DNA molecule of claim 21, further comprising a nucleotide sequence encoding a portion of an extracellular domain from ErbB3 that binds a ligand for ErbB3.

29. The recombinant DNA molecule of claim 21, further comprising a nucleotide sequence encoding amino acids 1-499 of the ErbB3 receptor.

30. The recombinant DNA molecule of claim 21, further comprising a nucleotide sequence encoding amino acids 1-531 of the ErbB3 receptor.

31. The recombinant DNA molecule of claim 21, further comprising a nucleotide sequence encoding amino acids 1-624 of the ErbB3 receptor.

32. The recombinant DNA molecule of claim 21, further comprising a nucleotide sequence encoding a portion of an extracellular domain from ErbB4 that binds a ligand for ErbB4.

33. The recombinant DNA molecule of claim 21, further comprising a nucleotide sequence encoding amino acids 1-496 of the ErbB4 receptor.

34. The recombinant DNA molecule of claim 21, further comprising a nucleotide sequence encoding amino acids 1-528 of the ErbB4 receptor.

35. The recombinant DNA molecule of claim 21, further comprising a nucleotide sequence encoding amino acids 1-626 of the ErbB4 receptor.

36. The recombinant DNA molecule of claim 21, further comprising a nucleotide sequence encoding a portion of an extracellular domain from ErbB1 that binds a ligand for ErbB1 and a portion of an extracellular domain from ErbB3 that binds a ligand for ErbB3.

37. The recombinant DNA molecule of claim 21, further comprising a nucleotide sequence encoding a portion of an extracellular domain from ErbB1 that binds a ligand for ErbB1 and a portion of an extracellular domain from ErbB4 that binds a ligand for ErbB4.

38. The recombinant DNA molecule of any of claims 32 and 33, wherein the nucleotide sequence encoding the carboxy-terminal ErbB ligand binding site encodes an amino acid sequence that is reversed in the amino to carboxy terminal direction.

39. The recombinant DNA molecule of any of claims 21-38, wherein the nucleotide sequence encodes a linker that joins the binding sites.

40. The recombinant DNA molecule of any of claims 21-38, wherein the nucleotide sequence further encodes a fusion partner.

41. A host cell comprising a recombinant DNA molecule encoding a protein having binding affinity for a first and a second ErbB ligand at separate binding sites in a single covalently joined protein molecule.

42. The host cell of claim 41, wherein the cell produces a bivalent binding molecule having binding affinity for a first and a second ErbB ligand at separate binding sites in a single covalently joined protein molecule.

43. The host cell of claim 41, wherein the cell transports a portion of the binding molecules to the exterior of the cell and into the surrounding media.

44. The host cell of claim 41, wherein the recombinant DNA molecule encodes a portion of an extracellular domain of an ErbB receptor that binds a ligand to an ErbB receptor.

45. The host cell of claim 41, wherein the recombinant DNA molecule encodes a portion of an extracellular domain of an ErbB1 receptor that binds a ligand to an ErbB1 receptor.

46. The host cell of claim 41, wherein the recombinant DNA molecule encodes a portion of an extracellular domain of an ErbB3 receptor that binds a ligand to an ErbB3 receptor.

47. The host cell of claim 41, wherein the recombinant DNA molecule encodes a portion of an extracellular domain of an ErbB4 receptor that binds a ligand to an ErbB4 receptor.

48. The host cell of claim 41, wherein the host cell is a eukaryotic cell.

49. The host cell of claim 41, wherein the host cell is a mammalian cell.

50. The host cell of claim 41, wherein the host cell is a CHO cell.

51. The host cell of claim 41, wherein the host cell is a yeast cell.

52. The host cell of claim 41, wherein the host cell is a prokaryotic cell.

53. A method for treating a disease comprising administering to a patient in need of treatment an effective amount of a bivalent binding molecule having binding affinity for a first and a second ErbB ligand at separate binding sites in a single covalently joined protein molecule.

54. The method for treating a disease of claim 53, wherein the binding molecule further comprises an extracellular domain of an ErbB receptor.

55. The method for treating a disease of claim 53, wherein the binding molecule further comprises an extracellular domain of ErbB1 that binds a ligand for ErbB1.

56. The method for treating a disease of claim 53, wherein the binding molecule further comprises an extracellular domain of ErbB3 that binds a ligand for ErbB3.

57. The method for treating a disease of claim 53, wherein the binding molecule further comprises an extracellular domain of ErbB4 that binds a ligand for ErbB4.

58. A method of diagnosing a cancer comprising contacting a tumor cell with a bivalent binding molecule having binding affinity for a first and a second ErbB ligand at separate binding sites in a single covalently joined protein molecule.

59. A binding molecule comprising a single molecule having affinity to EGF, TGFα, HB-EGF, Betacellulin, Amphiregulin, Epiregulin, Epigen, Neuregulin 1α, Neuregulin 1β, Neuregulin 2α, Neuregulin 2β, Neuregulin 3 and Neuregulin 4.

60. A method of treating a disease or condition which is improved, ameliorated, or inhibited by removal or inhibition of an ErbB ligand, comprising administering to a subject in need thereof a bivalent binding molecule having binding affinity for a first and a second ErbB ligand at separate binding sites in a single covalently joined protein molecule.


SEQUENCE LISTING (42 sequences)

SEQ ID NO: 1 - DNA, 1596 bp, Homo sapiens
ctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcata

SEQ ID NO: 2 - protein, 532 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECI

SEQ ID NO: 3 - DNA, 1596 bp, Homo sapiens
atatgcgagtctaacgaggtgtttgagaggccagagggtgagctgcttaactgcaaggacgtgtgcgaaaggggccgaagcgtcaatcggtgctctgtctgcgacaggcccgagccgggctggtgcggcgagccctcctgcttggcccattgcgtccagggcacagccaagtgcagcaacgaaggtagaaacagcataattaaaaccaaacagggttccaccgggtttctgaaaaaatggaacataacaaatgcatattgcttgaataaaaacggatcaattatagtggatggagatagtatagagaagctctcccgcttaggattgtccacaataaacctgagcgtcgtcgcactttcttttcagggtcatcaaaagaccaggggccgcataatcgaactaaacgagtttgcccatctcgacacgaggaacgaaccttgggctcagattctgttgtttgggacaatcgaaaaggtaaccaaactgattgatctggaacagccagatctgcctcctactcatacattctccgacggtaggtttgcagtgccgctgatccacctcgatggcagtatctccacctgcaacaaattccacaaaattaatacggctaatatatccctctcagacaaatttgaaggtattggtataggaaactgtgtgaaacgctgccctggggaatgcaagaagtgtaagcgcgtcggcgacgaagagatggagtatagcgacgccgggtgtgcccgagtctgctcgggccacgatacagtggtgtataatcgtccctgtaagaaggtgtgcaccgccggttttagctacaaaggcgagcccaacgtggatatgcagtacacgacccccaactacctcatgctcccaccctgcaccgacaagtgcacggccgaagaccgattcaaacgctgcgtcctgtgcgacagcgagcggcccggcacatgcggcgcagcttgtcagaaccactgctgcgacagtccctccaagggccgttgccgcgggtcctgccagcaggcctgtatcatcaaaaccctgaaacagtgcaacgaggagggagcaggttggtgcagcgggaatccctgtagcccagattgtaagcaatgcagcggcctgcacaaccagttcgacatgtcgatgaacagcctctttgacagtagcgtcatagaccggtggcagatcagcgaggtgaactgcctggcccctaacaacagcttccgggtggccggccatctgatcgaacagttaaatagaatgcccctggagaagctgggaaccaaaaatgcagattataactctttagtcgcattagcctattccaatgaatactacatgaatggaagaatcatccagctgaacgaattgcctattcgagaggtgacaaacctcgccattctcgtctatggtgctgtggagcagatcaccaagttattctcccttgattataataggcaggtgtataccattgaattgaatgggcttgtcgtggagtgtaacaatttcatgaggcagctcagcctctttcatgatgaatttactggcttgcagacgctcaagaacagtacgggccaatgcgttaaaaaggaagagctg

SEQ ID NO: 4 - protein, 532 aa, Homo sapiens
ICESNEVFERPEGELLNCKDVCERGRSVNRCSVCDRPEPGWCGEPSCLAHCVQGTAKCSNEGRNSIIKTKQGSTGFLKKWNITNAYCLNKNGSIIVDGDSIEKLSRLGLSTINLSVVALSFQGHQKTRGRIIELNEFAHLDTRNEPWAQILLFGTIEKVTKLIDLEQPDLPPTHTFSDGRFAVPLIHLDGSISTCNKFHKINTANISLSDKFEGIGIGNCVKRCPGECKKCKRVGDEEMEYSDAGCARVCSGHDTVVYNRPCKKVCTAGFSYKGEPNVDMQYTTPNYLMLPPCTDKCTAEDRFKRCVLCDSERPGTCGAACQNHCCDSPSKGRCRGSCQQACIIKTLKQCNEEGAGWCSGNPCSPDCKQCSGLHNQFDMSMNSLFDSSVIDRWQISEVNCLAPNNSFRVAGHLIEQLNRMPLEKLGTKNADYNSLVALAYSNEYYMNGRIIQLNELPIREVTNLAILVYGAVEQITKLFSLDYNRQVYTIELNGLVVECNNFMRQLSLFHDEFTGLQTLKNSTGQCVKKEEL

SEQ ID NO: 5 - DNA, 1593 bp, Homo sapiens
tccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttc

SEQ ID NO: 6 - protein, 531 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECF

SEQ ID NO: 7 - DNA, 1584 bp, Homo sapiens
cagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtg

SEQ ID NO: 8 - protein, 528 aa, Homo sapiens
QSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICV

SEQ ID NO: 9 - DNA, 72 bp, Homo sapiens
atgcgaccctccgggacggccggggcagcgctcctggcgctgctggctgcgctctgcccggcgagtcgggct

SEQ ID NO: 10 - protein, 24 aa, Homo sapiens
MRPSGTAGAALLALLAALCPASRA

SEQ ID NO: 11 - DNA, 72 bp, Homo sapiens
atgggaccctccgggacggccggggcagcgctcctggcgctgctggctgcgctctgcccggcgagtcgggct

SEQ ID NO: 12 - protein, 24 aa, Homo sapiens
MGPSGTAGAALLALLAALCPASRA

SEQ ID NO: 13 - DNA, 57 bp, Homo sapiens
atgagggcgaacgacgctctgcaggtgctgggcttgcttttcagcctggcccggggc

SEQ ID NO: 14 - protein, 19 aa, Homo sapiens
MRANDALQVLGLLFSLARG

SEQ ID NO: 15 - DNA, 57 bp, Homo sapiens
atgggggcgaacgacgctctgcaggtgctgggcttgcttttcagcctggcccggggc

SEQ ID NO: 16 - protein, 19 aa, Homo sapiens
MGANDALQVLGLLFSLARG

SEQ ID NO: 17 - DNA, 75 bp, Homo sapiens
atgaagccggcgacaggactttgggtctgggtgagccttctcgtggcggcggggaccgtccagcccagcgattct

SEQ ID NO: 18 - protein, 25 aa, Homo sapiens
MKPATGLWVWVSLLVAAGTVQPSDS

SEQ ID NO: 19 - DNA, 75 bp, Homo sapiens
atggggccggcgacaggactttgggtctgggtgagccttctcgtggcggcggggaccgtccagcccagcgattct

SEQ ID NO: 20 - protein, 25 aa, Homo sapiens
MGPATGLWVWVSLLVAAGTVQPSDS

SEQ ID NO: 21 - DNA, 1500 bp, Homo sapiens
ctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtc

SEQ ID NO: 22 - protein, 500 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCV

SEQ ID NO: 23 - DNA, 1497 bp, Homo sapiens
tccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttg

SEQ ID NO: 24 - protein, 499 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCL

SEQ ID NO: 25 - DNA, 1488 bp, Homo sapiens
cagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctg

SEQ ID NO: 26 - protein, 496 aa, Homo sapiens
QSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCL

SEQ ID NO: 27 - protein, 7 aa, Tobacco etch virus
  MISC_FEATURE (7)..(7): Xaa can be Gly or Ser
ETVRFQX

SEQ ID NO: 28 - DNA, 60 bp, Tobacco etch virus
ctagagaaaacctgtacttccagtcccatcatcatcatcatcattgagcggccgcgggcc

SEQ ID NO: 29 - protein, 15 aa, Artificial
  Linker sequence encodes
GGGGSGGGGSGGGGS

SEQ ID NO: 30 - DNA, 52 bp, Tobacco etch virus
cgcggccgctcaatgatgatgatgatgatgggactggaagtacaggttttct

SEQ ID NO: 31 - DNA, 38 bp, Homo sapiens
agctgctagcgccaccatgcgaccctccgggacggccg

SEQ ID NO: 32 - DNA, 38 bp, Homo sapiens
agctgctagcgccaccatgaagccggcgacaggacttt

SEQ ID NO: 33 - DNA, 85 bp, Homo sapiens
tctggtacccgatccgccaccgccagagccacctccgcctgaaccgcctccaccaccggtgacgcagtccctgggctccgggccc

SEQ ID NO: 34 - DNA, 85 bp, Homo sapiens
tctggtacccgatccgccaccgccagagccacctccgcctgaaccgcctccaccaccggtcagacattggtctggcccaggtccc

SEQ ID NO: 35 - DNA, 40 bp, Homo sapiens
agcgctagcgccaccatgagggcgaacgacgctctgcagg

SEQ ID NO: 36 - DNA, 34 bp, Homo sapiens
agcaccggtcaagcactgaccagggcctgggccc

SEQ ID NO: 37 - DNA, 31 bp, Homo sapiens
cggggtaccctggaggaaaagaaagtttgcc

SEQ ID NO: 38 - DNA, 34 bp, Homo sapiens
cggggtacctccgaggtgggcaactctcaggcag

SEQ ID NO: 39 - DNA, 31 bp, Homo sapiens
cggggtacccagtcagtgtgtgcaggaacgg

SEQ ID NO: 40 - DNA, 31 bp, Homo sapiens
tgctctagagacgcagtccctgggctccggg

SEQ ID NO: 41 - DNA, 34 bp, Homo sapiens
tgctctagacaagcactgaccagggcctgggccc

SEQ ID NO: 42 - DNA, 31 bp, Homo sapiens
tgctctagacagacattggtctggcccaggt